(19) United States
(12) Patent Application Publication
(10) Pub. No.: US 2025/0017850 A1
(43) Pub. Date: Jan. 16, 2025

(54) SALBUTAMOL DELIVERY FORMULATIONS, DEVICES AND METHODS

(71) Applicant: HONEYWELL INTERNATIONAL INC, Charlotte, NC (US)

(72) Inventors: ERIK BOLDT, Buffalo, NY (US); GREGORY LAURENCE SMITH, Buffalo, NY (US)

(73) Assignee: HONEYWELL INTERNATIONAL INC., Charlotte, NC (US)

(21) Appl. No.: 18/771,332

(22) Filed: Jul. 12, 2024

Related U.S. Application Data
(60) Provisional application No. 63/526,827, filed on Jul. 14, 2023.

(51) Int. Cl. A61K 9/00; A61K 31/137; A61K 47/06; A61K 47/10; A61K 47/12

(52) U.S. Cl. CPC A61K 9/008; A61K 31/137; A61K 47/06; A61K 47/10; A61K 47/12

(57) ABSTRACT

Disclosed are medicinal compositions, and devices, methods and systems which use same, comprising a propellant and at least one medicinally active compound, said propellant comprising at least 1234ze(E) and ethanol.

### CROSS REFERENCE TO RELATED APPLICATIONS

This application is related to and claims the priority benefit of U.S. Provisional Application 63/526,827, filed Jul. 14, 2023, which is incorporated herein by reference in its entirety.

### FIELD OF THE INVENTION

This invention relates to medicament delivery compositions, systems, devices and methods. In particular aspects, this invention relates to medicinal aerosol formulations, methods and devices used for metered dose delivery of salbutamol (i.e., albuterol) or a pharmaceutically acceptable salt or ester thereof (e.g., salbutamol sulfate).

### BACKGROUND OF THE INVENTION

Metered dose inhalers (MDIs) have long been used to deliver medicaments, such as bronchodilator drugs and steroids, to the areas of patients needing treatment. Compared with oral administration of bronchodilators, inhalation therapy using MDIs frequently has the advantage of relatively rapid onset of action and relatively low instance of systemic side effects.

Asthma is described as a chronic disease that involves inflammation of the pulmonary airways and bronchial hyperresponsiveness that results in the clinical expression of a lower airway obstruction that usually is reversible. The pathophysiology of asthma or related disorders involves bronchoconstriction resulting from bronchial smooth muscle spasm and airway inflammation with mucosal edema. Treatment of asthma and other related disorders (including Chronic Obstructive Pulmonary Disease (COPD)) has included the administration of β-2 agonists, also known as, β-2 adrenoreceptor agonists. Such β-2 adrenoreceptor agonists are known to provide a bronchodilator effect to patients, resulting in relief from the symptoms of breathlessness. More particularly, β-2 adrenoreceptor agonists have been shown to increase the conductance of potassium channels in airway muscle cells, leading to membrane hyperpolarization and relaxation. Salbutamol is a short-acting β-2 adrenoreceptor and has been recommended and used for the relief of acute asthma symptoms.

MDIs are one of the most widely used system for the delivery of drugs via inhalation. The ultimate objective of MDIs is to accurately deliver, upon actuation by the person in need of relief, a specific predetermined amount of a drug to the respiratory tract of a patient using a delivery composition in which the drug is dissolved, suspended, or dispersed. The delivery composition will generally include, in addition to the active ingredient, a propellant. For a propellant to function satisfactorily in MDIs, it needs to have a number of properties. These include an appropriate boiling point and vapor pressure so that it can be liquefied in a closed container at room temperature but develop a high enough pressure when the MDI is activated to deliver the drug as an atomized formulation even at low ambient temperatures. Further, the propellant should be of low acute and chronic toxicity. It should have a high degree of chemical stability in contact with the drug, the container and the metallic and non-metallic components of the MDI device, and have a low propensity to extract low molecular weight substances from any elastomeric materials in the MDI device. The propellant preferably also is able to maintaining the drug in a homogeneous solution, in a stable suspension or in a stable dispersion for a sufficient time to permit reproducible delivery of the drug in use. When the drug is in suspension in the propellant, the density of the liquid propellant is desirably similar to that of the solid drug in order to avoid rapid sinking or floating of the drug particles in the liquid. Finally, the propellant should not present a significant flammability risk to the patient in use. In particular, it should form a non-flammable or low flammability mixture when mixed with air in the respiratory tract.

The propellants which have heretofore commonly used include a mixture of liquefied chlorofluorocarbons (CFC's) selected to have the vapor pressure necessary to produce the desired propulsive force while at the same time providing stability of the medicament formulation and the other properties mentioned above. Methane and ethane series CFCs, such tetrachloromethane (CFC-11), trichlorofluoromethane (CFC-12) and 1,2 dichlorotetrafluoroethane (CFC-114), have commonly been used as propellants in aerosol formulations for inhalation administration.

The use of CFCs has environmental drawbacks. It is now known that CFC's tend to react with the ozone layer around the earth and thereby result in some level of ozone depletion. As a result various governmental and international organizations have been engaged in efforts to reduce or eliminate the use of CFCs. The volume of CFCs which have been used in connection with MDIs may be considered low compared to other uses, such as refrigerants and blowing agents. Nevertheless, a potential ozone depletion advantage may be achieved by reducing or eliminating CFCs from MDIs and other medicament delivery systems.

Because of the potential damage to the earth's ozone layer caused by chlorine-containing compounds (such as chlorofluorocarbons (CFCs), hydrochlorofluorocarbons (HCFCs) and the like), there has thus been an increasing need for new fluorocarbon and hydrofluorocarbon compounds and compositions that offer alternatives with reduced ozone depletion potential. For example, efforts are under way to replace chlorine-containing propellants with non-chlorine-containing compounds that will not deplete the ozone layer, such as hydrofluorocarbons (HFCs).

U.S. Pat. No. 5,776,434—Purewal, et al. has recognized the ozone depletion problem of CFCs and has proposed the use of a non-chlorine containing compound, namely, 1,1,1,2-tetrafluoroethane (sometimes referred to herein as HFA-134a or HFC-134a) as a propellant for medicinal aerosol formulations when used in combination with a surface active agent and an adjuvant having a higher polarity than 1,1,1,2-tetrafluoroethane. However, in 1998 the International Programme on Chemical Safety (IPCS), published a Concise International Chemical Assessment Document (No. 11) indicating that 1,1,1,2-tetrafluorethane has a significant global warming potential.

HFC-227ea (1,1,1,2,3,3,3-heptafluoropropane) has also been proposed as low ozone depletion potential substitute for CFCs in MDIs. However, this compound also has a significant global warming potential.

U.S. Pat. No. 9,308,199, which is assigned to the assignee of the present application, describes the use of fluoroolefins, preferably hydrofluorolefins (HFO), as medicinally acceptable carriers that are able to overcome the environmental deficiencies of CFCs, HFCs and HCFCs mentioned above. Tetrafluoropropenes, including 1,3,3,3-tetrafluoropropene (HFO-1234ze) and 2,3,3,3-tetrafluoropropene (HFO-1234yf) are disclosed as being preferred.

WO2023/039103 mentions that HFOs have been proposed as propellants for MDIs but also notes that no MDI product has been successfully developed or commercialized using HFOs as a propellant. The '103 publication discloses an MDI that uses a formulation comprising greater than 70% by weight of HFO-1234ze(E), ethanol and at least one active pharmaceutical ingredient (API).

Notwithstanding the disclosures as mentioned above, applicants have come to recognize the need for delivery compositions, systems, devices and methods for albuterol or a pharmaceutically acceptable salt or ester thereof that at once provide relatively low ozone depletion potential, relatively low global warming potential and the ability to maintaining the API in a homogeneous solution, in a stable suspension or in a stable dispersion for a sufficient time to permit reproducible accurate delivery of the drug in use.

### SUMMARY

Applicants have found that many of the shortcomings of the prior compositions can be overcome and/or that many of the above-noted needs can be satisfied by pharmaceutical compositions of the present invention and the use thereof in MDIs and inhalation delivery methods.

The present invention also includes pharmaceutical compositions comprising:
a. an active pharmaceutical ingredient (API) comprising salbutamol;
b. from about 85% by weight to about 99% by weight of HFO-1234ze(E);
c. from 1% by weight to about 15% by weight of ethanol; and
d. from greater than 0.001% by weight to less than 0.1% by weight of oleic acid.
   For the purposes of convenience, pharmaceutical compositions according to this paragraph are referred to as Pharmaceutical Composition 1A.

The present invention also includes pharmaceutical compositions comprising:
a. an active pharmaceutical ingredient (API) comprising salbutamol;
b. from about 85% by weight to about 99% by weight of HFO-1234ze(E);
c. from 1% by weight to about 15% by weight of ethanol; and
d. from greater than 0.005% by weight to less than 0.1% by weight of oleic acid.
   For the purposes of convenience, pharmaceutical compositions according to this paragraph are referred to as Pharmaceutical Composition 1 B.

The present invention also includes pharmaceutical compositions comprising:
a. an active pharmaceutical ingredient (API) comprising salbutamol;
b. from about 80% by weight to about 99% by weight of HFO-1234ze(E);
c. from 1% by weight to about 20% by weight of ethanol; and
d. from greater than 0.001% by weight to less than 0.1% by weight of oleic acid.
   For the purposes of convenience, pharmaceutical compositions according to this paragraph are referred to as Pharmaceutical Composition 1C.

The present invention also includes pharmaceutical compositions comprising:
a. an active pharmaceutical ingredient (API) comprising salbutamol;
b. from about 90% by weight to about 99% by weight of HFO-1234ze(E);
c. from 1% by weight to about 10% by weight of ethanol; and
d. from greater than 0.001% by weight to less than 0.1% by weight of oleic acid.
   For the purposes of convenience, pharmaceutical compositions according to this paragraph are referred to as Pharmaceutical Composition 1D.

The present invention also includes pharmaceutical compositions comprising:
a. an active pharmaceutical ingredient (API) comprising salbutamol;
b. from about 95% by weight to about 99% by weight of HFO-1234ze(E);
c. from 1% by weight to about 5% by weight of ethanol; and
d. from greater than 0.001% by weight to less than 0.1% by weight of oleic acid.
   For the purposes of convenience, pharmaceutical compositions according to this paragraph are referred to as Pharmaceutical Composition 1 E.

The present invention also includes pharmaceutical compositions comprising:
a. an active pharmaceutical ingredient (API) comprising salbutamol;
b. from about 85% by weight to about 99% by weight of HFO-1234ze(E);
c. from 1% by weight to about 15% by weight of ethanol; and
d. from greater than 0.01% by weight to less than 0.1% by weight of oleic acid.
   For the purposes of convenience, pharmaceutical compositions according to this paragraph are referred to as Pharmaceutical Composition 2A.

The present invention also includes pharmaceutical compositions comprising:
a. an active pharmaceutical ingredient (API) comprising salbutamol;
b. from about 80% by weight to about 99% by weight of HFO-1234ze(E);
c. from 1% by weight to about 20% by weight of ethanol; and
d. from greater than 0.01% by weight to less than 0.1% by weight of oleic acid.
   For the purposes of convenience, pharmaceutical compositions according to this paragraph are referred to as Pharmaceutical Composition 2B.

The present invention also includes pharmaceutical compositions comprising:
a. an active pharmaceutical ingredient (API) comprising salbutamol;
b. from about 90% by weight to about 99% by weight of HFO-1234ze(E);
c. from 1% by weight to about 10% by weight of ethanol; and
d. from greater than 0.01% by weight to less than 0.1% by weight of oleic acid.
   For the purposes of convenience, pharmaceutical compositions according to this paragraph are referred to as Pharmaceutical Composition 2C.

The present invention also includes pharmaceutical compositions comprising:
a. an active pharmaceutical ingredient (API) comprising salbutamol;
b. from about 95% by weight to about 99% by weight of HFO-1234ze(E);
c. from 1% by weight to about 5% by weight of ethanol; and
d. from greater than 0.01% by weight to less than 0.1% by weight of oleic acid.
   For the purposes of convenience, pharmaceutical compositions according to this paragraph are referred to as Pharmaceutical Composition 2D.

The present invention also includes pharmaceutical compositions comprising:
a. an active pharmaceutical ingredient (API) comprising salbutamol;
b. from about 85% by weight to about 99% by weight of HFO-1234ze(E);
c. from 1% by weight to about 15% by weight of ethanol; and
d. from greater than 0.01% by weight to 0.05% by weight of oleic acid.
   For the purposes of convenience, pharmaceutical compositions according to this paragraph are referred to as Pharmaceutical Composition 3A.

The present invention also includes pharmaceutical compositions comprising:
a. an active pharmaceutical ingredient (API) comprising salbutamol;
b. from about 80% by weight to about 99% by weight of HFO-1234ze(E);
c. from 1% by weight to about 20% by weight of ethanol; and
d. from greater than 0.01% by weight to 0.05% by weight of oleic acid.
   For the purposes of convenience, pharmaceutical compositions according to this paragraph are referred to as Pharmaceutical Composition 3B.

The present invention also includes pharmaceutical compositions comprising:
a. an active pharmaceutical ingredient (API) comprising salbutamol;
b. from about 90% by weight to about 99% by weight of HFO-1234ze(E);
c. from 1% by weight to about 10% by weight of ethanol; and
d. from greater than 0.01% by weight to 0.05% by weight of oleic acid.
   For the purposes of convenience, pharmaceutical compositions according to this paragraph are referred to as Pharmaceutical Composition 3C.

The present invention also includes pharmaceutical compositions comprising:
a. an active pharmaceutical ingredient (API) comprising salbutamol;
b. from about 95% by weight to about 99% by weight of HFO-1234ze(E);
c. from 1% by weight to about 5% by weight of ethanol; and
d. from greater than 0.01% by weight to 0.05% by weight of oleic acid.
   For the purposes of convenience, pharmaceutical compositions according to this paragraph are referred to as Pharmaceutical Composition 3D.

The present invention also includes pharmaceutical compositions comprising:
a. an active pharmaceutical ingredient (API) comprising salbutamol;
b. from about 80% by weight to about 99% by weight of HFO-1234ze(E);
c. from 1% by weight to about 10% by weight of ethanol; and
d. from greater than 0.01% by weight to 0.05% by weight of oleic acid.
   For the purposes of convenience, pharmaceutical compositions according to this paragraph are referred to as Pharmaceutical Composition 3B.

The present invention also includes pharmaceutical compositions comprising:
a. an active pharmaceutical ingredient (API) comprising salbutamol;
b. from about 85% by weight to about 96.5% by weight of HFO-1234ze(E);
c. from 3.5% by weight to about 15% by weight of ethanol; and
d. from greater than 0.005% by weight to less than 0.1% by weight of oleic acid.
   For the purposes of convenience, pharmaceutical compositions according to this paragraph are referred to as Pharmaceutical Composition 4.

The present invention also includes pharmaceutical compositions comprising:
a. an active pharmaceutical ingredient (API) comprising salbutamol;
b. from about 85% by weight to about 96.5% by weight of HFO-1234ze(E);
c. from 3.5% by weight to about 15% by weight of ethanol; and
d. from greater than 0.01% by weight to less than 0.1% by weight of oleic acid.
   For the purposes of convenience, pharmaceutical compositions according to this paragraph are referred to as Pharmaceutical Composition 5.

The present invention also includes pharmaceutical compositions comprising:
a. an active pharmaceutical ingredient (API) comprising salbutamol;
b. from about 85% by weight to about 96.5% by weight of HFO-1234ze(E);
c. from 3.5% by weight to about 15% by weight of ethanol; and
d. from greater than 0.01% by weight to 0.05% by weight of oleic acid.
   For the purposes of convenience, pharmaceutical compositions according to this paragraph are referred to as Pharmaceutical Composition 6.

The present invention also includes pharmaceutical compositions comprising:
a. an active pharmaceutical ingredient (API) comprising salbutamol;
b. from about 85% by weight to about 90% by weight of HFO-1234ze(E);
c. from 10% by weight to about 15% by weight of ethanol; and
d. from greater than 0.005% by weight to less than 0.1% by weight of oleic acid.
   For the purposes of convenience, pharmaceutical compositions according to this paragraph are referred to as Pharmaceutical Composition 7.

The present invention also includes pharmaceutical compositions comprising:
a. an active pharmaceutical ingredient (API) comprising salbutamol;
b. from about 85% by weight to about 90% by weight of HFO-1234ze(E);
c. from 10% by weight to about 15% by weight of ethanol; and
d. from greater than 0.01% by weight to less than 0.1% by weight of oleic acid.
   For the purposes of convenience, pharmaceutical compositions according to this paragraph are referred to as Pharmaceutical Composition 8.

The present invention also includes pharmaceutical compositions comprising:
a. an active pharmaceutical ingredient (API) comprising salbutamol;
b. from about 85% by weight to about 90% by weight of HFO-1234ze(E);
c. from 10% by weight to about 15% by weight of ethanol; and
d. from greater than 0.01% by weight to 0.05% by weight of oleic acid.
   For the purposes of convenience, pharmaceutical compositions according to this paragraph are referred to as Pharmaceutical Composition 9.

The present invention also includes pharmaceutical compositions comprising:
a. an active pharmaceutical ingredient (API) comprising salbutamol;
b. about 85% by weight of HFO-1234ze(E);
c. about 15% by weight of ethanol; and
d. from greater than 0.005% by weight to less than 0.1% by weight of oleic acid.
   For the purposes of convenience, pharmaceutical compositions according to this paragraph are referred to as Pharmaceutical Composition 10.

The present invention also includes pharmaceutical compositions comprising:
a. an active pharmaceutical ingredient (API) comprising salbutamol;
b. about 85% by weight of HFO-1234ze(E);
c. about 15% by weight of ethanol; and
d. from greater than 0.01% by weight to less than 0.1% by weight of oleic acid.
   For the purposes of convenience, pharmaceutical compositions according to this paragraph are referred to as Pharmaceutical Composition 11.

The present invention also includes pharmaceutical compositions comprising:
a. an active pharmaceutical ingredient (API) comprising salbutamol;
b. about 90% by weight of HFO-1234ze(E);
c. about 10% by weight of ethanol; and
d. from greater than 0.01% by weight to 0.05% by weight of oleic acid.
   For the purposes of convenience, pharmaceutical compositions according to this paragraph are referred to as Pharmaceutical Composition 12.

The present invention also includes pharmaceutical compositions comprising:
a. an active pharmaceutical ingredient (API) comprising salbutamol;
b. from about 85% by weight to less than 95% by weight of HFO-1234ze(E); and
c. from greater than 5% by weight to about 15% by weight of ethanol.
   For the purposes of convenience, pharmaceutical compositions according to this paragraph are referred to as Pharmaceutical Composition 13.

The present invention also includes pharmaceutical compositions comprising:
a. an active pharmaceutical ingredient (API) comprising salbutamol;
b. about 85% by weight of HFO-1234ze(E); and
c. about 15% by weight of ethanol.
   For the purposes of convenience, pharmaceutical compositions according to this paragraph are referred to as Pharmaceutical Composition 14.

### BRIEF DESCRIPTION OF THE DRAWING

The invention will now be described with reference to the accompanying drawings in which:

FIG. 1 is a cross-sectional side view of an inhaler including a canister containing a valve according to the present disclosure.

FIG. 2 is a detailed cross-sectional side view of the inhaler of FIG. 1.

FIG. 3 is a cross-sectional side view of a metering valve for an inhaler.

FIGS. Ex2A and Ex2B are charts illustrating data from Example 2.

FIG. ExC3 is a chart illustrating data from Comparative Example 3.

FIG. Ex3 is a chart illustrating data from Example 3.

FIGS. Ex4A-Ex4C are charts illustrating data from Example 4.

FIG. Ex5 is a chart illustrating data from Example 5.

### DETAILED DESCRIPTION OF PREFERRED EMBODIMENTS

### I. Definitions

For the purposes of this invention, the term “about” in relation to the amounts expressed in weight percent for amounts greater than 2% means that the amount of the component can vary by an amount of +/−2% by weight.

For the purposes of this invention, the term “about” in relation to the amounts expressed in weight percent for amounts less than 2% and greater than 1% means that the amount of the component can vary by an amount of +/−1% by weight.

For the purposes of this invention, the term “about” in relation to the amounts expressed in weight percent for amounts less than 1% and greater than 0.5% means that the amount of the component can vary by an amount of +/−0.2% by weight.

For the purposes of this invention, the term “about” in relation to the amounts expressed in weight percent for amounts less than 0.5% means that the amount of the component can vary by an amount of +/−0.05% by weight.

For the purposes of this invention, the term “about” in relation to temperatures in degrees centigrade (° C.) means that the stated temperature can vary by an amount of +/−5° C.

The terms “HFC-134a” and “R134a” means 1,1,1,2-tetrafluoroethane.

The terms “HFO1234ze(E),” R1234ze(E) and “1234ze(E)” as used herein each mean trans-1,3,3,3-tetrafluoropropene. Unless otherwise stated, “HFO1234ze,” R1234ze and “1234ze” mean trans-1,3,3,3-tetrafluoropropene.

The term “salbutamol” as used herein encompasses any and all pharmaceutically acceptable versions of salbutamol, including salts of salbutamol.

The term “salbutamol sulfate” as used herein encompasses any and all pharmaceutically acceptable versions sulfate salts salbutamol.

Reference herein to a group of defined items includes all such defined items, including all such items with suffix designations.

### II. The Compositions

The preferred pharmaceutical compositions of the present invention, including each of Pharmaceutical Formulations 1-14, are stable suspensions or dispersions of the API in a carrier comprising the other required components of the formulation, including particularly the HFO-1234ze(E) and ethanol.

### A. Concentration of Components

The concentration of the components in the present compositions can generally vary widely within the broad scope of the present invention. The concentration of the API contained in the compositions of the present invention, including each of Pharmaceutical Formulations 1-14, is preferably from 0.0008% to 3.4% by weight, or 0.01% to 1.0% by weight, or from 0.05% to 0.5%. Preferred compositions include those identified in the following Table 1, with all numerical values understood to be preceded by “about” and with the following designations in the table having the following meanings: “comp” means that the formulation comprises the identified components; CEO means that the formulation consists essentially of the identified components; CO means that the formulation consists of the identified components; TSI means the Trubiscan Stability Index at 30 seconds as determined in accordance with the examples hereof; and NR means the component is not required to be present.

| Pharmaceutical | | Components, wt % | | | | Property |
|---|---|---|---|---|---|---|
| Formulation No. | | 1234ze(E) | Ethanol | Oleic Acid | Salbutamol | TSI |
| PC15A | Comp | 85-99 | 1-15 | 0.005-0.1 | 0.1-0.5 | NR |
| PC15B | CEO | 85-99 | 1-15 | 0.005-0.1 | 0.1-0.5 | NR |
| PC15C | CO | 85-99 | 1-15 | 0.005-0.1 | 0.1-0.5 | NR |
| PC15D | Comp | 85-99 | 1-15 | 0.005-0.1 | 0.1-0.5 | <10 |
| PC15E | CEO | 85-99 | 1-15 | 0.005-0.1 | 0.1-0.5 | <10 |
| PC15F | CO | 85-99 | 1-15 | 0.005-0.1 | 0.1-0.5 | <10 |
| PC15G | Comp | 85-99 | 1-15 | 0.005-0.1 | 0.1-0.5 | <7 |
| PC15H | CEO | 85-99 | 1-15 | 0.005-0.1 | 0.1-0.5 | <7 |
| PC15I | CO | 85-99 | 1-15 | 0.005-0.1 | 0.1-0.5 | <7 |
| PC16A | Comp | 85-99 | 1-15 | 0.01-0.1 | 0.1-0.5 | NR |
| PC16B | CEO | 85-99 | 1-15 | 0.01-0.1 | 0.1-0.5 | NR |
| PC16C | CO | 85-99 | 1-15 | 0.01-0.1 | 0.1-0.5 | NR |
| PC16D | Comp | 85-99 | 1-15 | 0.01-0.1 | 0.1-0.5 | <10 |
| PC16E | CEO | 85-99 | 1-15 | 0.01-0.1 | 0.1-0.5 | <10 |
| PC16F | CO | 85-99 | 1-15 | 0.01-0.1 | 0.1-0.5 | <10 |
| PC16G | Comp | 85-99 | 1-15 | 0.01-0.1 | 0.1-0.5 | <7 |
| PC16H | CEO | 85-99 | 1-15 | 0.01-0.1 | 0.1-0.5 | <7 |
| PC16I | CO | 85-99 | 1-15 | 0.01-0.1 | 0.1-0.5 | <7 |
| PC17A | Comp | 85-99 | 1-15 | 0.01-0.05 | 0.1-0.5 | NR |
| PC17B | CEO | 85-99 | 1-15 | 0.01-0.05 | 0.1-0.5 | NR |
| PC17C | CO | 85-99 | 1-15 | 0.01-0.05 | 0.1-0.5 | NR |
| PC17D | Comp | 85-99 | 1-15 | 0.01-0.05 | 0.1-0.5 | <10 |
| PC17E | CEO | 85-99 | 1-15 | 0.01-0.05 | 0.1-0.5 | <10 |
| PC17F | CO | 85-99 | 1-15 | 0.01-0.05 | 0.1-0.5 | <10 |
| PC17G | Comp | 85-99 | 1-15 | 0.01-0.05 | 0.1-0.5 | <7 |
| PC17H | CEO | 85-99 | 1-15 | 0.01-0.05 | 0.1-0.5 | <7 |
| PC17I | CO | 85-99 | 1-15 | 0.01-0.05 | 0.1-0.5 | <7 |
| PC18A | Comp | 85-96.5 | 3.5-15 | 0.005-0.1 | 0.1-0.5 | NR |
| PC18B | CEO | 85-96.5 | 3.5-15 | 0.005-0.1 | 0.1-0.5 | NR |
| PC18C | CO | 85-96.5 | 3.5-15 | 0.005-0.1 | 0.1-0.5 | NR |
| PC18D | Comp | 85-96.5 | 3.5-15 | 0.005-0.1 | 0.1-0.5 | <10 |
| PC18E | CEO | 85-96.5 | 3.5-15 | 0.005-0.1 | 0.1-0.5 | <10 |
| PC18F | CO | 85-96.5 | 3.5-15 | 0.005-0.1 | 0.1-0.5 | <10 |
| PC18G | Comp | 85-96.5 | 3.5-15 | 0.005-0.1 | 0.1-0.5 | <7 |
| PC18H | CEO | 85-96.5 | 3.5-15 | 0.005-0.1 | 0.1-0.5 | <7 |
| PC18I | CO | 85-96.5 | 3.5-15 | 0.005-0.1 | 0.1-0.5 | <7 |
| PC19A | Comp | 85-96.5 | 3.5-15 | 0.01-0.1 | 0.1-0.5 | NR |
| PC19B | CEO | 85-96.5 | 3.5-15 | 0.01-0.1 | 0.1-0.5 | NR |
| PC19C | CO | 85-96.5 | 3.5-15 | 0.01-0.1 | 0.1-0.5 | NR |
| PC19D | Comp | 85-96.5 | 3.5-15 | 0.01-0.1 | 0.1-0.5 | <10 |
| PC19E | CEO | 85-96.5 | 3.5-15 | 0.01-0.1 | 0.1-0.5 | <10 |
| PC19F | CO | 85-96.5 | 3.5-15 | 0.01-0.1 | 0.1-0.5 | <10 |
| PC19G | Comp | 85-96.5 | 3.5-15 | 0.01-0.1 | 0.1-0.5 | <7 |
| PC19H | CEO | 85-96.5 | 3.5-15 | 0.01-0.1 | 0.1-0.5 | <7 |
| PC19I | CO | 85-96.5 | 3.5-15 | 0.01-0.1 | 0.1-0.5 | <7 |
| PC20A | Comp | 85-96.5 | 3.5-15 | 0.01-0.05 | 0.1-0.5 | NR |
| PC20B | CEO | 85-96.5 | 3.5-15 | 0.01-0.05 | 0.1-0.5 | NR |
| PC20C | CO | 85-96.5 | 3.5-15 | 0.01-0.05 | 0.1-0.5 | NR |
| PC20D | Comp | 85-96.5 | 3.5-15 | 0.01-0.05 | 0.1-0.5 | <10 |
| PC20E | CEO | 85-96.5 | 3.5-15 | 0.01-0.05 | 0.1-0.5 | <10 |
| PC20F | CO | 85-96.5 | 3.5-15 | 0.01-0.05 | 0.1-0.5 | <10 |
| PC20G | Comp | 85-96.5 | 3.5-15 | 0.01-0.05 | 0.1-0.5 | <7 |
| PC20H | CEO | 85-96.5 | 3.5-15 | 0.01-0.05 | 0.1-0.5 | <7 |
| PC20I | CO | 85-96.5 | 3.5-15 | 0.01-0.05 | 0.1-0.5 | <7 |
| PC21A | Comp | 85-<95 | >5-15 | 0.005-0.1 | 0.1-0.5 | NR |
| PC21B | CEO | 85-<95 | >5-15 | 0.005-0.1 | 0.1-0.5 | NR |
| PC21C | CO | 85-<95 | >5-15 | 0.005-0.1 | 0.1-0.5 | NR |
| PC21D | Comp | 85-<95 | >5-15 | 0.005-0.1 | 0.1-0.5 | <10 |
| PC21E | CEO | 85-<95 | >5-15 | 0.005-0.1 | 0.1-0.5 | <10 |
| PC21F | CO | 85-<95 | >5-15 | 0.005-0.1 | 0.1-0.5 | <10 |
| PC21G | Comp | 85-<95 | >5-15 | 0.005-0.1 | 0.1-0.5 | <7 |
| PC21H | CEO | 85-<95 | >5-15 | 0.005-0.1 | 0.1-0.5 | <7 |
| PC21I | CO | 85-<95 | >5-15 | 0.005-0.1 | 0.1-0.5 | <7 |
| PC22A | Comp | 85-<95 | >5-15 | 0.01-0.1 | 0.1-0.5 | NR |
| PC22B | CEO | 85-<95 | >5-15 | 0.01-0.1 | 0.1-0.5 | NR |
| PC22C | CO | 85-<95 | >5-15 | 0.01-0.1 | 0.1-0.5 | NR |
| PC22D | Comp | 85-<95 | >5-15 | 0.01-0.1 | 0.1-0.5 | <10 |
| PC22E | CEO | 85-<95 | >5-15 | 0.01-0.1 | 0.1-0.5 | <10 |
| PC22F | CO | 85-<95 | >5-15 | 0.01-0.1 | 0.1-0.5 | <10 |
| PC22G | Comp | 85-<95 | >5-15 | 0.01-0.1 | 0.1-0.5 | <7 |
| PC22H | CEO | 85-<95 | >5-15 | 0.01-0.1 | 0.1-0.5 | <7 |
| PC22I | CO | 85-<95 | >5-15 | 0.01-0.1 | 0.1-0.5 | <7 |
| PC23A | Comp | 85-<95 | >5-15 | 0.01-0.05 | 0.1-0.5 | NR |
| PC23B | CEO | 85-<95 | >5-15 | 0.01-0.05 | 0.1-0.5 | NR |
| PC23C | CO | 85-<95 | >5-15 | 0.01-0.05 | 0.1-0.5 | NR |
| PC23D | Comp | 85-<95 | >5-15 | 0.01-0.05 | 0.1-0.5 | <10 |
| PC23E | CEO | 85-<95 | >5-15 | 0.01-0.05 | 0.1-0.5 | <10 |
| PC23F | CO | 85-<95 | >5-15 | 0.01-0.05 | 0.1-0.5 | <10 |
| PC23G | Comp | 85-<95 | >5-15 | 0.01-0.05 | 0.1-0.5 | <7 |
| PC23H | CEO | 85-<95 | >5-15 | 0.01-0.05 | 0.1-0.5 | <7 |
| PC23I | CO | 85-<95 | >5-15 | 0.01-0.05 | 0.1-0.5 | <7 |
| PC24A | Comp | 85-99 | 1-15 | 0.005-0.1 | 0.25-0.35 | NR |
| PC24B | CEO | 85-99 | 1-15 | 0.005-0.1 | 0.25-0.35 | NR |
| PC24C | CO | 85-99 | 1-15 | 0.005-0.1 | 0.25-0.35 | NR |
| PC24D | Comp | 85-99 | 1-15 | 0.005-0.1 | 0.25-0.35 | <10 |
| PC24E | CEO | 85-99 | 1-15 | 0.005-0.1 | 0.25-0.35 | <10 |
| PC24F | CO | 85-99 | 1-15 | 0.005-0.1 | 0.25-0.35 | <10 |
| PC24G | Comp | 85-99 | 1-15 | 0.005-0.1 | 0.25-0.35 | <7 |
| PC24H | CEO | 85-99 | 1-15 | 0.005-0.1 | 0.25-0.35 | <7 |
| PC24I | CO | 85-99 | 1-15 | 0.005-0.1 | 0.25-0.35 | <7 |
| PC25A | Comp | 85-99 | 1-15 | 0.01-0.1 | 0.25-0.35 | NR |
| PC25B | CEO | 85-99 | 1-15 | 0.01-0.1 | 0.25-0.35 | NR |
| PC25C | CO | 85-99 | 1-15 | 0.01-0.1 | 0.25-0.35 | NR |
| PC25D | Comp | 85-99 | 1-15 | 0.01-0.1 | 0.25-0.35 | <10 |
| PC25E | CEO | 85-99 | 1-15 | 0.01-0.1 | 0.25-0.35 | <10 |
| PC25F | CO | 85-99 | 1-15 | 0.01-0.1 | 0.25-0.35 | <10 |
| PC25G | Comp | 85-99 | 1-15 | 0.01-0.1 | 0.25-0.35 | <7 |
| PC25H | CEO | 85-99 | 1-15 | 0.01-0.1 | 0.25-0.35 | <7 |
| PC25I | CO | 85-99 | 1-15 | 0.01-0.1 | 0.25-0.35 | <7 |
| PC26A | Comp | 85-99 | 1-15 | 0.01-0.05 | 0.25-0.35 | NR |
| PC26B | CEO | 85-99 | 1-15 | 0.01-0.05 | 0.25-0.35 | NR |
| PC26C | CO | 85-99 | 1-15 | 0.01-0.05 | 0.25-0.35 | NR |
| PC26D | Comp | 85-99 | 1-15 | 0.01-0.05 | 0.25-0.35 | <10 |
| PC26E | CEO | 85-99 | 1-15 | 0.01-0.05 | 0.25-0.35 | <10 |
| PC26F | CO | 85-99 | 1-15 | 0.01-0.05 | 0.25-0.35 | <10 |
| PC26G | Comp | 85-99 | 1-15 | 0.01-0.05 | 0.25-0.35 | <7 |
| PC26H | CEO | 85-99 | 1-15 | 0.01-0.05 | 0.25-0.35 | <7 |
| PC26I | CO | 85-99 | 1-15 | 0.01-0.05 | 0.25-0.35 | <7 |
| PC27A | Comp | 85-96.5 | 3.5-15 | 0.005-0.1 | 0.25-0.35 | NR |
| PC27B | CEO | 85-96.5 | 3.5-15 | 0.005-0.1 | 0.25-0.35 | NR |
| PC27C | CO | 85-96.5 | 3.5-15 | 0.005-0.1 | 0.25-0.35 | NR |
| PC27D | Comp | 85-96.5 | 3.5-15 | 0.005-0.1 | 0.25-0.35 | <10 |
| PC27E | CEO | 85-96.5 | 3.5-15 | 0.005-0.1 | 0.25-0.35 | <10 |
| PC27F | CO | 85-96.5 | 3.5-15 | 0.005-0.1 | 0.25-0.35 | <10 |
| PC27G | Comp | 85-96.5 | 3.5-15 | 0.005-0.1 | 0.25-0.35 | <7 |
| PC27H | CEO | 85-96.5 | 3.5-15 | 0.005-0.1 | 0.25-0.35 | <7 |
| PC27I | CO | 85-96.5 | 3.5-15 | 0.005-0.1 | 0.25-0.35 | <7 |
| PC28A | Comp | 85-96.5 | 3.5-15 | 0.01-0.1 | 0.25-0.35 | NR |
| PC28B | CEO | 85-96.5 | 3.5-15 | 0.01-0.1 | 0.25-0.35 | NR |
| PC28C | CO | 85-96.5 | 3.5-15 | 0.01-0.1 | 0.25-0.35 | NR |
| PC28D | Comp | 85-96.5 | 3.5-15 | 0.01-0.1 | 0.25-0.35 | <10 |
| PC28E | CEO | 85-96.5 | 3.5-15 | 0.01-0.1 | 0.25-0.35 | <10 |
| PC28F | CO | 85-96.5 | 3.5-15 | 0.01-0.1 | 0.25-0.35 | <10 |
| PC28G | Comp | 85-96.5 | 3.5-15 | 0.01-0.1 | 0.25-0.35 | <7 |
| PC28H | CEO | 85-96.5 | 3.5-15 | 0.01-0.1 | 0.25-0.35 | <7 |
| PC28I | CO | 85-96.5 | 3.5-15 | 0.01-0.1 | 0.25-0.35 | <7 |
| PC29A | Comp | 85-96.5 | 3.5-15 | 0.01-0.05 | 0.25-0.35 | NR |
| PC29B | CEO | 85-96.5 | 3.5-15 | 0.01-0.05 | 0.25-0.35 | NR |
| PC29C | CO | 85-96.5 | 3.5-15 | 0.01-0.05 | 0.25-0.35 | NR |
| PC29D | Comp | 85-96.5 | 3.5-15 | 0.01-0.05 | 0.25-0.35 | <10 |
| PC29E | CEO | 85-96.5 | 3.5-15 | 0.01-0.05 | 0.25-0.35 | <10 |
| PC29F | CO | 85-96.5 | 3.5-15 | 0.01-0.05 | 0.25-0.35 | <10 |
| PC29G | Comp | 85-96.5 | 3.5-15 | 0.01-0.05 | 0.25-0.35 | <7 |
| PC29H | CEO | 85-96.5 | 3.5-15 | 0.01-0.05 | 0.25-0.35 | <7 |
| PC29I | CO | 85-96.5 | 3.5-15 | 0.01-0.05 | 0.25-0.35 | <7 |
| PC30A | Comp | 85-<95 | >5-15 | 0.005-0.1 | 0.25-0.35 | NR |
| PC30B | CEO | 85-<95 | >5-15 | 0.005-0.1 | 0.25-0.35 | NR |
| PC30C | CO | 85-<95 | >5-15 | 0.005-0.1 | 0.25-0.35 | NR |
| PC30D | Comp | 85-<95 | >5-15 | 0.005-0.1 | 0.25-0.35 | <10 |
| PC30E | CEO | 85-<95 | >5-15 | 0.005-0.1 | 0.25-0.35 | <10 |
| PC30F | CO | 85-<95 | >5-15 | 0.005-0.1 | 0.25-0.35 | <10 |
| PC30G | Comp | 85-<95 | >5-15 | 0.005-0.1 | 0.25-0.35 | <7 |
| PC30H | CEO | 85-<95 | >5-15 | 0.005-0.1 | 0.25-0.35 | <7 |
| PC30I | CO | 85-<95 | >5-15 | 0.005-0.1 | 0.25-0.35 | <7 |
| PC31A | Comp | 85-<95 | >5-15 | 0.01-0.1 | 0.25-0.35 | NR |
| PC31B | CEO | 85-<95 | >5-15 | 0.01-0.1 | 0.25-0.35 | NR |
| PC31C | CO | 85-<95 | >5-15 | 0.01-0.1 | 0.25-0.35 | NR |
| PC31D | Comp | 85-<95 | >5-15 | 0.01-0.1 | 0.25-0.35 | <10 |
| PC31E | CEO | 85-<95 | >5-15 | 0.01-0.1 | 0.25-0.35 | <10 |
| PC31F | CO | 85-<95 | >5-15 | 0.01-0.1 | 0.25-0.35 | <10 |
| PC31G | Comp | 85-<95 | >5-15 | 0.01-0.1 | 0.25-0.35 | <7 |
| PC31H | CEO | 85-<95 | >5-15 | 0.01-0.1 | 0.25-0.35 | <7 |
| PC31I | CO | 85-<95 | >5-15 | 0.01-0.1 | 0.25-0.35 | <7 |
| PC32A | Comp | 85-<95 | >5-15 | 0.01-0.05 | 0.25-0.35 | NR |
| PC32B | CEO | 85-<95 | >5-15 | 0.01-0.05 | 0.25-0.35 | NR |
| PC32C | CO | 85-<95 | >5-15 | 0.01-0.05 | 0.25-0.35 | NR |
| PC32D | Comp | 85-<95 | >5-15 | 0.01-0.05 | 0.25-0.35 | <10 |
| PC32E | CEO | 85-<95 | >5-15 | 0.01-0.05 | 0.25-0.35 | <10 |
| PC32F | CO | 85-<95 | >5-15 | 0.01-0.05 | 0.25-0.35 | <10 |
| PC32G | Comp | 85-<95 | >5-15 | 0.01-0.05 | 0.25-0.35 | <7 |
| PC32H | CEO | 85-<95 | >5-15 | 0.01-0.05 | 0.25-0.35 | <7 |
| PC32I | CO | 85-<95 | >5-15 | 0.01-0.05 | 0.25-0.35 | <7 |

For all compositions of the present invention, other than those defined as “consisting of” the designated components, including each of Pharmaceutical Compositions 1-32, the salbutamol comprises a salbutamol sulfate, or consists essentially of salbutamol sulfate, or consists of salbutamol sulfate.

For all compositions of the present invention, other than those defined as “consisting of” the designated components, including each of Pharmaceutical Compositions 1-32, additional components or excipients may be present. These components may have various uses and functions, including, but not limited to, facilitating formation of a suspension, stabilizing a suspension, and/or aiding in chemical stabilization of API or other components.

For all compositions of the present invention, including each of Pharmaceutical Compositions 1-32, the composition comprises a “suspension” or “dispersion” of the indicated API in the HFO-1234ze(E) and ethanol, and oleic acid when present. In all such compositions, including each of Pharmaceutical Compositions 1-32, the designated API is in a microparticulate solid form (preferably micronized, but it can also be size-reduced by a multitude of other particle size reduction techniques). As used herein, a suspension/dispersion of an API comprises particles of the API that impart a visual impact to the unaided human eye, although there may also be a small amount of solubilized particulate material within the composition. For suspension formulations, including each of Pharmaceutical Formulations 1-32 solubilization of API is generally undesirable. In embodiments, including each of Pharmaceutical Formulations 1-32, there is minimal solubilization of the API, and in some preferred embodiments including each of Pharmaceutical Formulations 1-32, there is essentially no solubilization of the API.

The preferred pharmaceutical compositions of the present invention, including each of Pharmaceutical Formulations 1-32, have a degree of physical stability which avoids significant separation of the physical mixture via sedimentation or creaming of the suspended/dispersed particles.

In certain preferred forms, the compositions of the present invention, including each of Pharmaceutical Compositions 1-32, have a Global Warming Potential (GWP) of not greater than about 1500, more preferably not greater than about 75, and even more preferably not greater than about 10. As used herein, “GWP” is measured relative to that of carbon dioxide and over a 100-year time horizon, as defined in “The Scientific Assessment of Ozone Depletion, 2002, a report of the World Meteorological Association's Global Ozone Research and Monitoring Project,” which is incorporated herein by reference.

In certain preferred forms, the present compositions also preferably have an Ozone Depletion Potential (ODP) of not greater than 0.05, more preferably not greater than 0.02 and even more preferably about zero. As used herein, “ODP” is as defined in “The Scientific Assessment of Ozone Depletion, 2002, A report of the World Meteorological Association's Global Ozone Research and Monitoring Project,” which is incorporated herein by reference.

Many embodiments of the present invention, including each of Pharmaceutical Compositions 1-32, and particularly those in which the composition is in the form of a suspension, emulsion or dispersion, preferably include a stabilizing agent. Stabilizing agents for such suspensions, emulsions and dispersions are well known, and it is contemplated that all such stabilizing agents are adaptable for use in accordance with the present invention. Exemplary stabilizing agents include, either alone or in combination, hydrochloric acid, sulphuric acid, nitric acid, phosphoric acid, ascorbic acid, citric acid, benzalkonium chloride, ethylene diamine tetraacetic, and pharmacologically tolerable salts thereof. Although it is contemplated that the stabilizing agents of the present mentioned it may be included in the compositions in widely varying amounts, it is generally preferred in many embodiments that the stabilizing agent is present in an amount of from about 40 to about 100 ppm by weight of the composition.

### III. Devices and Methods

The present invention includes devices for the delivery by inhalation the composition of the present invention, including each of Pharmaceutical Compositions 1-32. In certain preferred embodiments, the devices of the present invention comprise a container, preferably an aerosol canister, containing a pressurized formulation of the present invention, including each of Pharmaceutical Compositions 1-32, and preferably having a metered dose dispensing valve operable between non-dispensing and dispensing positions. The present devices preferably also comprise an actuator, which in preferred embodiments comprises a housing adapted to receive the aerosol container and to define a chamber in fluid communication with a patient port for introducing the medicament into the oral and/or nasal cavity of the patient, preferably in the form of a mouthpiece and/or nasal adapter. The actuator also preferably includes a nozzle block adapted to receive the valve stem of the dispensing valve, the nozzle block preferably comprising a passage in fluid communication with the valve stem and terminating in an orifice for directing medicament from the valve stem into the chamber.

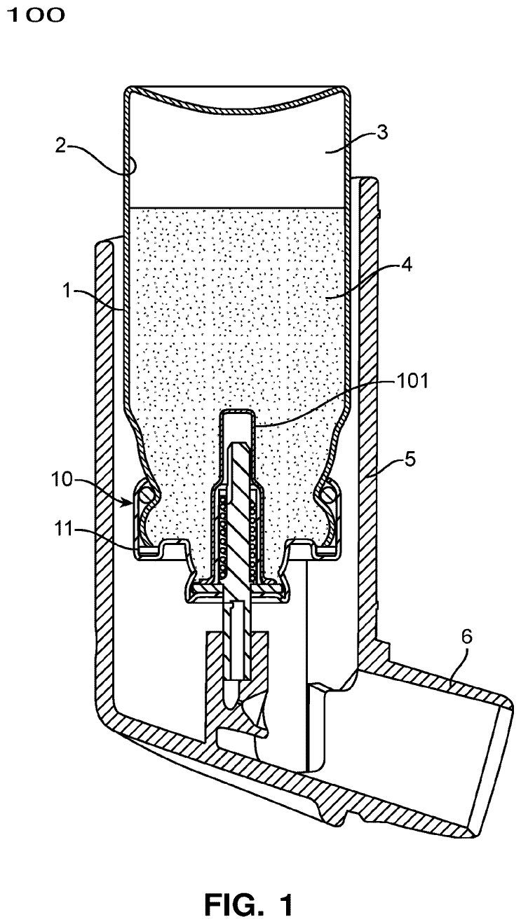

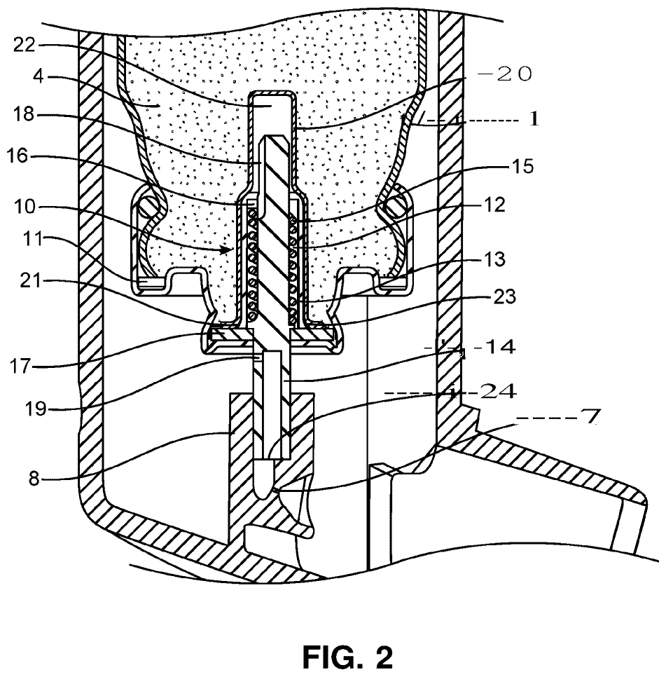

By way of example but not by way of limitation, FIG. 1 shows one embodiment of a metered dose inhaler 100, including an aerosol canister 1 fitted with a metered dose metering valve 10 (shown in its resting position). The metering valve 10 is typically affixed, i.e., crimped, onto the canister via a cap or ferrule 11 (typically made of aluminum or an aluminum alloy) which is generally provided as part of the valve assembly. Between the canister and the ferrule there may be one or more seals. In the embodiments shown in FIG. 1 and FIG. 2 between the canister 1 and the ferrule 11 there are two seals including, e.g., an O-ring seal and a gasket seal.

As shown in FIG. 1, the canister/valve dispenser is typically provided with an actuator 5 including an appropriate patient port 6, such as a mouthpiece. For administration to the nasal cavities the patient port is generally provided in an appropriate form (e.g., smaller diameter tube, often sloping upwardly) for delivery through the nose. Actuators are generally made of a plastic material, for example polypropylene or polyethylene. As can be seen from FIG. 1, inner walls 2 of the canister and outer walls 101 of the portion(s) of the metering valve 10 located within the canister define a formulation chamber 3 in which aerosol formulation 4 is contained.

The valve 10 shown in FIG. 1 and FIG. 2, includes a metering chamber 12, defined in part by an inner valve body 13, through which a valve stem 14 passes. The valve stem 14, which is biased outwardly by a compression spring 15, is in sliding sealing engagement with an inner tank seal 16 and an outer diaphragm seal 17. The valve 10 also includes a second valve body 20 in the form of a bottle emptier. The inner valve body 13 (also referred to as the “primary” valve body) defines in part the metering chamber 12. The second valve body 20 (also referred to as the “secondary” valve body) defines in part a pre-metering region or chamber besides serving as a bottle emptier.

Referring to FIG. 2, aerosol formulation 4 can pass from the formulation chamber 3 into a pre-metering chamber 22 provided between the secondary valve body 20 and the primary valve body 13 through an annular space 21 between a flange 23 of the secondary valve body 20 and the primary valve body 13. To actuate (fire) the valve 10, the valve stem 14 is pushed inwardly relative to the canister 1 from its resting position shown in FIG. 1 and FIG. 2, allowing formulation to pass from the metering chamber 12 through a side hole 19 in the valve stem and through a stem outlet 24 to an actuator nozzle 7 then out to the patient. When the valve stem 14 is released, formulation enters into the valve 10, in particular into the pre-metering chamber 22, through the annular space 21 and thence from the pre-metering chamber through a groove 18 in the valve stem past the tank seal 16 into the metering chamber 12.

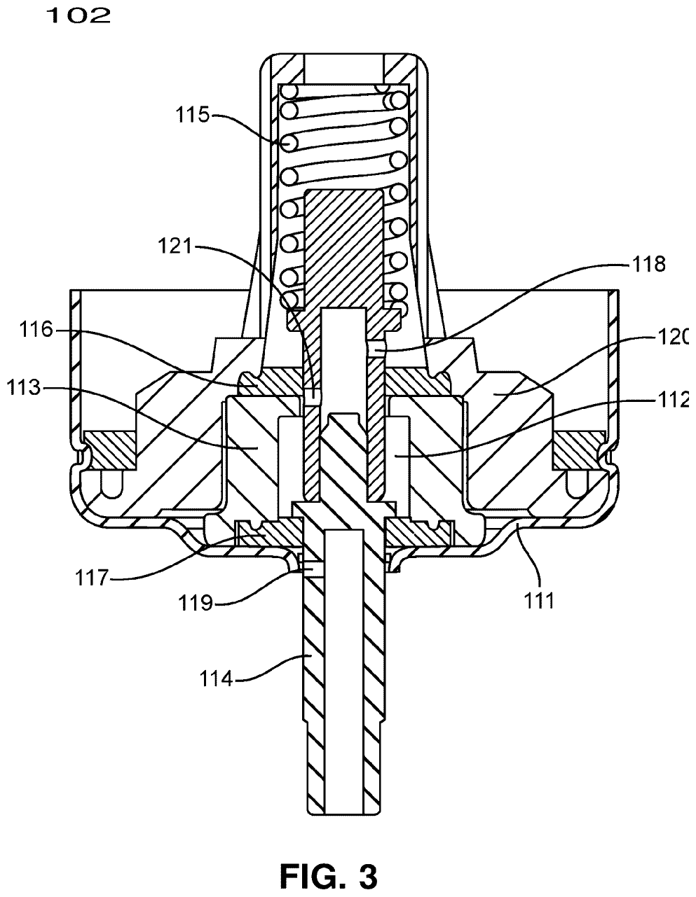

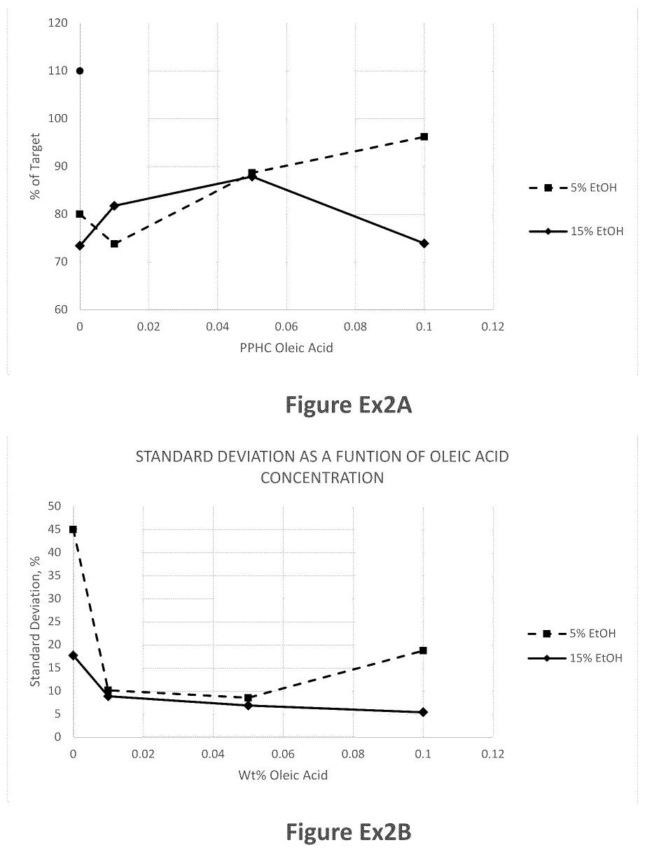

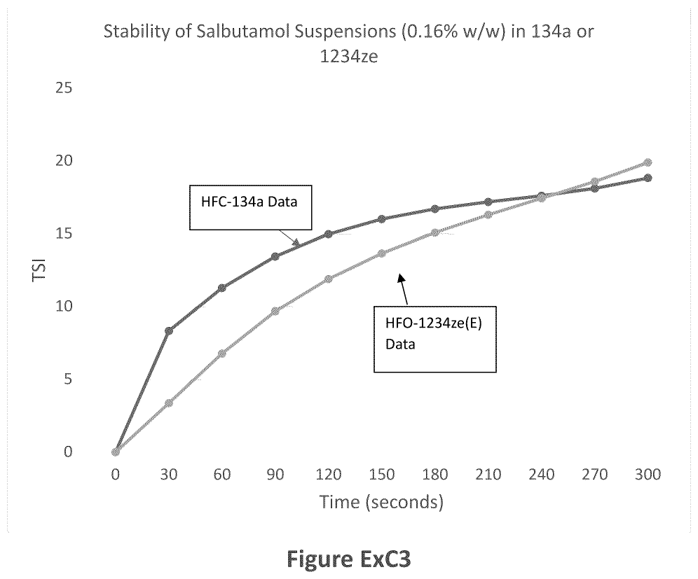

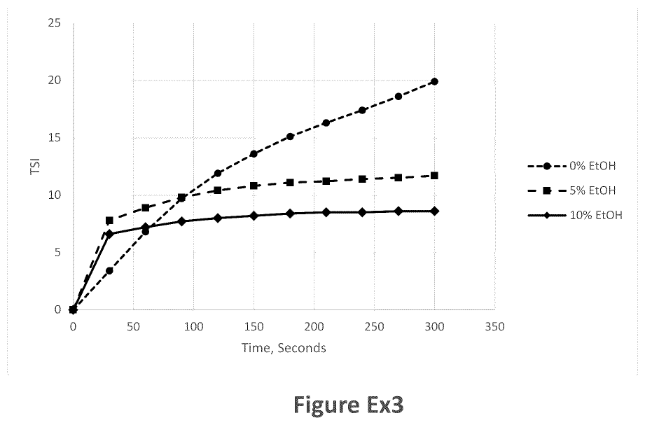

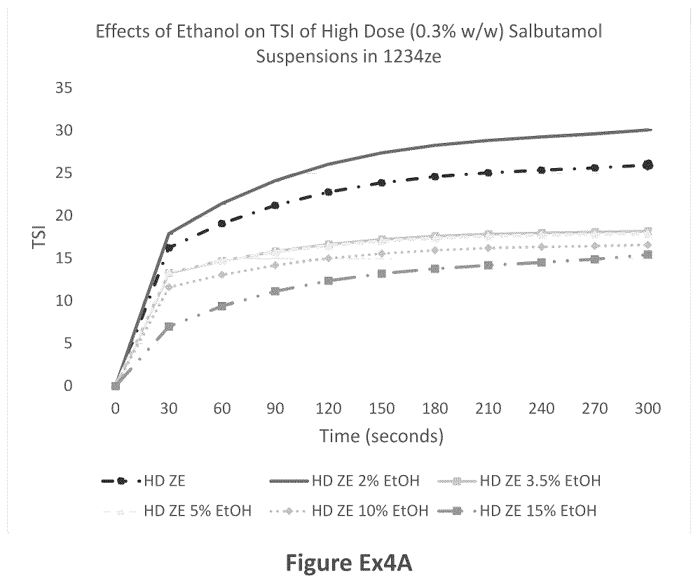

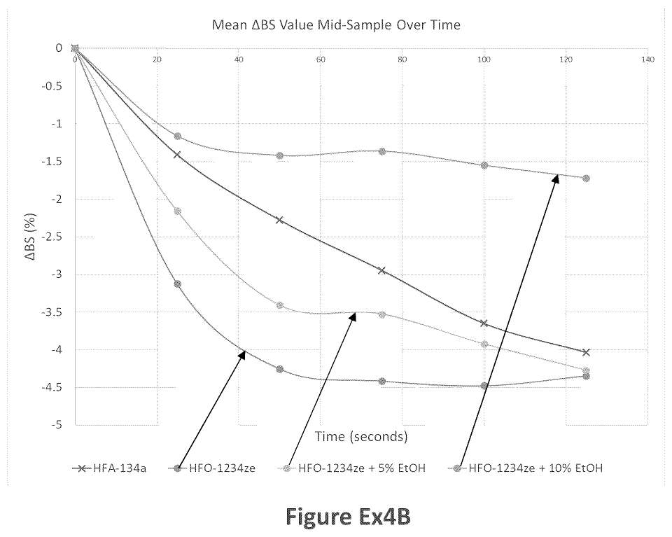

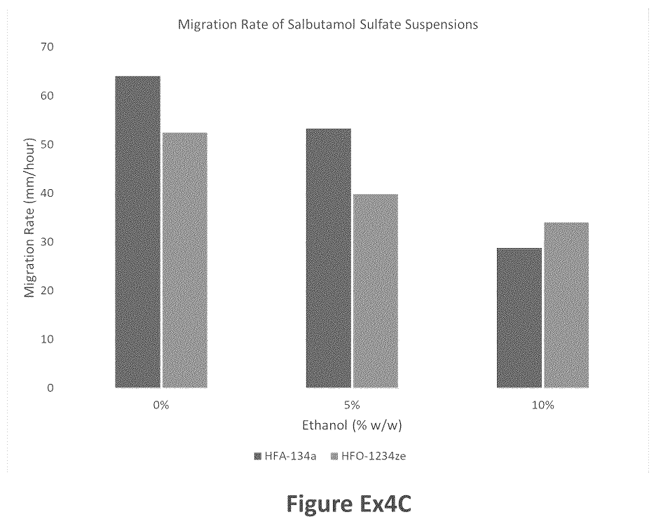

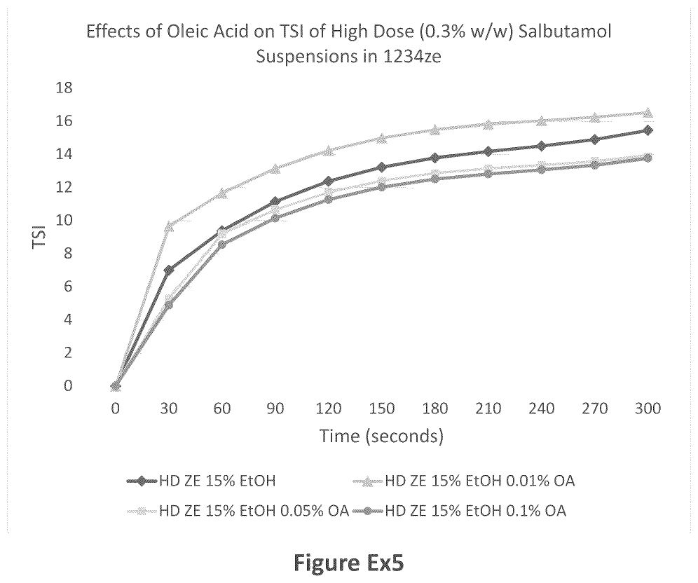

FIG. 3 shows another embodiment of a metered dose aerosol metering valve 102, different from the embodiment shown in FIG. 1 and FIG. 2, in its rest position. The valve 102 has a metering chamber 112 defined in part by a metering tank 113 through which a stem 114 is biased outwardly by spring 115. The stem 114 is made in two parts that are push fit together before being assembled into the valve 102. The stem 114 has an inner seal 116 and an outer seal 117 disposed about it and forming sealing contact with the metering tank 113. A valve body 120 crimped into a ferrule 111 retains the aforementioned components in the valve. In use, formulation enters the metering chamber via orifices 121 and 118. The formulation's outward path from the metering chamber 112 when a dose is dispensed is via orifice 119.

In certain embodiments the invention device is constructed such that airflow due to patient inhalation is prevented or reduced in the vicinity of the orifice at all times or only during dispensing of the medicament from the valve. Either of such arrangements has the effect of substantially reducing the velocity of the emitted spray compared to an inhaler which allows free flow of air in the vicinity of the nozzle block during dispensing of the medicament.

In certain embodiments, the actuator is constructed such that the distance from the nozzle to the mouthpiece is from approximately 1 to 15 cm, preferably 4 to 6 cm, with a chamber/mouthpiece diameter from 1 to 4 cm, 0.5 to 1 cm in the case of a nasal adapter.

In certain preferred but non-limiting embodiments, the actuator possess air inlets which enable the patient to inhale though the patient port, preferably without encountering significant resistance since the patient may have breathing difficulties when taking the medication, for example, during an asthma attack. However, the air inlets, for example in the mouthpiece, preferably do not concentrate the airflow into an area that is too narrow, as this will give a high velocity of incoming air which will deflect the spray onto the wall of the mouthpiece opposite the air inlets. In certain preferred embodiments the air inlets are positioned downstream of the nozzle, in the region of the turbulent zone and/or downstream of the turbulent zone. The positioning and direction of the air inlets may also affect the deposition of medicament within the chamber and mouthpiece. In one arrangement air inlets comprise a series of holes and optionally may be interdispersed with fluid deflection structures on the wall of the chamber, to direct air into the turbulent zone to mix air with the aerosol stream. Further, the mouthpiece may be constructed of porous material to allow a multiplicity of finely divided air vents to provide air flow over a larger surface area.

In certain embodiments the actuator possesses air inlets upstream of or in the vicinity of the nozzle but the air inlets are blocked when the valve is fired to release the aerosol spray. The air inlets are opened after the spray has been released by which time the velocity of the stream will have been reduced and the turbulent zone formed. Upon inhalation, an airflow is established from the air inlets to the mouthpiece which entrains the residual aerosol spray. The actuator may include additional air inlets downstream of the nozzle, as described above with respect to the first embodiment. These downstream air inlets do not need to close during release of the aerosol spray.

In certain embodiments, a porous membrane is present to introduce air into or downstream of the turbulent zone. One advantage of the use of such a membrane is that the air is introduced more uniformly and diffusely around the circumference of the spray, thereby acting as a buffer between the turbulent flow and the wall. The effect is to reduce drug deposition in the device. The membrane may optionally be protected from dirt or contact by the user's lips by an additional part of the mouthpiece. When present, it is preferred that the porous membrane material (50) must not significantly impede the patient's ability to inhale through the device. A suitable material is Whatmann No. 4 filter paper; but other materials may be used, such as those used in cylindrical air filters or membrane filters, or such as those formed by sintering polymers. A preferred porous membrane material is in the form of a cylinder made by fusing together small pellets of polypropylene.

For certain medicaments, it is preferred to configure the device so as to reduce contact between the medicament and parts of the patient's body that it is not intended to contact. For example, residues of the medicament deposited on internal surfaces of actuators may be fingered and transferred to other body parts. In such cases, the device may be configured to include one or more fluid flow deflectors to allow the spray to pass through, whilst limiting access by the patient to internal surfaces of the actuator. Of course, the device may be configured for intranasal delivery. This is normally quite undesirable, since the medicaments were designed for delivery to the respiratory system and may not have an appropriate effect when deposited in the oropharynx and allowed to enter the digestive tract. In an effort to overcome this problem, certain embodiments of the present device include the provision of a holding volume, commonly called a spacer, in which the medicament is fired. The spacer preferably allows the velocity of the medicament to be reduced and may also allow some propellant evaporation to occur. Spacers can improve the performance of a metered dose inhaler by reducing oropharyngeal deposition.

The total amount of composition of the present invention, including each of Pharmaceutical Compositions 1-32, contained in the canister preferably is selected so that at least a portion of the propellant in the canister is present as a liquid after a predetermined number of medicinal doses have been delivered. The predetermined number of doses may be 5 to 200, 30 to 200, 60 to 200, 60 to 120, 60, 120, 200, or any other number of doses. In preferred embodiments, the total amount of composition of the present invention, including each of Pharmaceutical Compositions 1-32, in the canister may be from 1.0 grams (g) to 30.0 g, 2.0 g to 20.0 g, or 5.0 to 10.0 g. The total amount of composition of the present invention, including each of Pharmaceutical Compositions 1-32, is typically selected to be greater than the product of the predetermined number of doses and the metering volume of the metering valve. In some embodiments, the total amount of composition is greater than 1.1 times, greater than 1.2 times, greater than 1.3 times, greater than 1.4 times, or greater than 1.5 times the product of the predetermined number of doses and the metering volume of the metering valve. This helps to ensures that the amount of each dose remains relatively constant through the life of the inhaler.

The present invention thus provide inhalers, and preferably metered dose inhalers (MDIs) for the treatment of asthma and other chronic obstructive pulmonary diseases and for delivery of pharmaceutical compositions, including each of Pharmaceutical Compositions 1-32, to accessible mucous membranes or intranasally. The present invention thus includes methods for delivering of pharmaceutical compositions, including each of Pharmaceutical Compositions 1-32, for purpose of treating ailments, diseases and similar health related problems of an organism (such as a human or animal) comprising applying a composition of the present invention containing a medicament or other therapeutic component to the organism in need of treatment. In certain preferred embodiments, the step of applying the present composition comprises providing an MDI containing the composition of the present invention, including each of Pharmaceutical Compositions 1-32, and then discharging the present composition from the MDI.

The amount of API that is delivered may be determined by the required dose per actuation and the MDI metering valve size, that is, the size of the metering chamber, which may be between 5 microliters (μL or mci) and 200 microliters, between 25 microliters and 200 microliters, between 25 microliters and 150 microliters, between 25 microliters and 100 microliters, or between 25 microliters and 65 microliters.

In certain embodiments, typical formulations of the present disclosure include the API in an amount of at least 0.001 milligram per actuation (mg/actuation) (1 microgram (μg) per actuation), or at least 0.01 mg/actuation (10 μg/actuation). In certain embodiments, typical formulations of the present disclosure include the API in an amount of less than 0.5 mg/actuation (500 μg/actuation).

In embodiments, typical formulations of the present disclosure include the API in an amount of at least 1 μg/actuation, at least 10 μg/actuation, at least 50 μg/actuation, at least 100 μg/actuation, at least 150 μg/actuation, at least 200 μg/actuation, at least 300 μg/actuation, or at least 400 μg/actuation. In embodiments, typical formulations of the present disclosure include the API in an amount of less than 500 μg/actuation, at most 400 μg/actuation, at most 300 μg/actuation or at most 200 μg/actuation. In some preferred embodiments, formulations of the present disclosure include the API in an amount of 80 μg/actuation to 200 μg/actuation.

The present invention includes methods of forming pharmaceutical compositions having improved stability, including each of Pharmaceutical Compositions 1-32, comprising forming a carrier comprising not more than 99% by weight HFO-1234ze and from greater than about 0% by weight to less than about 10% by weight of ethanol, and suspending in said carrier an API comprising, consisting essentially of, or consisting of salbutamol, wherein said suspension has improved migration rates compared to carrier comprising greater than about 10% of ethanol.

The present invention also includes methods of forming pharmaceutical compositions having improved stability, including each of Pharmaceutical Compositions 1-32, comprising forming a carrier comprising not more than 99% by weight of HFO-1234ze and from greater than about 0% by weight to less than about 10% by weight of ethanol and suspending in said carrier an API comprising, consisting essentially of, or consisting of from 0.25 wt % to about 0.5 wt % salbutamol, wherein said suspension has improved migration rates compared to carrier comprising greater than about 10% of ethanol.

The present invention includes methods of forming pharmaceutical compositions having improved stability, including each of Pharmaceutical Compositions 1-32, comprising forming a carrier comprising not more than 95% by weight and from greater than about 5% by weight to about 15% by weight of ethanol, suspending in said carrier an API comprising, consisting essentially of, or consisting of salbutamol, wherein said suspension has improved TSI compared to carrier comprising not greater than 2.5% by weight of ethanol.

The present invention includes methods of forming pharmaceutical compositions having improved stability, including each of Pharmaceutical Compositions 1-32, comprising forming a carrier comprising not more than 95% by weight and from greater than about 5% by weight to about 15% by weight of ethanol, suspending in said carrier an API comprising, consisting essentially of, or consisting of from 0.25 wt % to about 0.5 wt % salbutamol, wherein said suspension has improved TSI compared to carrier comprising not greater than 2.5% by weight of ethanol.

### EXAMPLES

### Comparative Example 1: Delivered Dose of Salbutamol Sulfate Suspensions in 100% HFO-1234ze(E) and in a Mixture of 95% HFO-1234ze(E) and 5% Ethanol

Salbutamol sulfate (API) was suspended in HFC-1234ze(E) and a mixture of HFC-1234ze(E) and ethanol in amounts as indicated in Table C1 below. Each formulation was then loaded into an MDI with a target dosage delivery of 167 micrograms per actuation and then tested to determine the Average Delivered Dose Uniformity (ADDU) and Standard Deviation of ADDU (generally following the guidelines set forth in USP601—Inhalation and Nasal Drug Products: Aerosols, Sprays, and Powders—Performance Quality Tests The results of this test are reported in Table ExC1 below.

TABLE ExC1

| Example No. | | |
|---|---|---|
| Components | ExC1A | ExC1B |
| HFC-1234ze(E), wt % | 99.7 | 94.7 |
| Ethanol, wt % | 0 | 5 |
| API, wt % | 0.3 | 0.3 |
| Total | 100 | 100 |
| ADDU, % of Target | 108.6% | 80.3% |
| Standard Deviation of | 45% | 45% |
| ADDU % | | |

As is seen from test result reported in Table ExC1 above, the use of a carrier comprising 100% HFO-1234ze(E) and a carrier comprising about 95% HFO-1234ze(E) and 5% ethanol each produced an ADDU within the preferred result of +/−25%. However, in each case the standard deviation of the result was unacceptably high, that is, at a standard deviation level of 45%.

### Example 1: Delivered Dose of Salbutamol Sulfate Suspensions in 85% HFO-1234ze(E) and 15% Ethanol

Salbutamol sulfate (API) was suspended in a mixture of about 85% by weight of HFO-1234ze(E) and 15% by weight of ethanol. The formulation was then loaded into an MDI with a target dosage delivery of 167 micrograms per actuation and then tested to determine the Average Delivered Dose Uniformity (ADDU) and Standard Deviation of ADDU (generally following the guidelines set forth in USP601—Inhalation and Nasal Drug Products: Aerosols, Sprays, and Powders—Performance Quality Tests The results of this test are reported in Table Ex1 below.

TABLE Ex1

| Example No. | |
|---|---|
| Carrier Components | Ex1 |
| HFC-1234ze(E), wt % | 84.7 |
| Ethanol, wt % | 15 |
| API, wt % | 0.3 |
| ADDU, % of Target | 73.2% |
| Standard Deviation of | 17.7% |
| ADDU % | |

As is seen from test result reported in Table Ex1 above, applicants have unexpected found that the use of a carrier comprising about 85% by weight of HFO-1234ze(E) and 15% ethanol each produced a standard deviation of the result at a level of below 20%, which is dramatically improved compared to the 45% level of Examples ExC1A and ExC1B. In addition, this unexpected result is achieved while producing an ADDU value that is within only 2% of the preferred value of 75%.

### Example 2: Delivered Dose of Salbutamol Sulfate Suspensions in 85% HFO-1234ze(E) and 15% Ethanol with Oleic Acid

Salbutamol sulfate (API) was suspended in a mixture of about 85% by weight of HFO-1234ze(E) and 15% by weight of ethanol with oleic acid in amounts of 0.01%, 0.05% and 0.1% by weight. The formulation was then loaded into an MDI with a target dosage delivery of 167 micrograms per actuation and then tested to determine the Average Delivered Dose Uniformity (ADDU) and Standard Deviation of ADDU (generally following the guidelines set forth in USP601—Inhalation and Nasal Drug Products: Aerosols, Sprays, and Powders—Performance Quality Tests The results of this test, together with the results from Comparative Example 1 and Example 1, are reported in Table Ex2 below and illustrated in FIGS. Ex2A and Ex2B hereof.

TABLE Ex2

| | Example No. | | | | | | | | |
|---|---|---|---|---|---|---|---|---|---|
| | ExC | ExC | | | | | | | |
| | 1A | 1B | Ex1 | Ex2A | Ex2B | Ex2C | Ex2D | Ex2E | Ex2F |
| Carrier | | | | | | | | | |
| Components | | | | | | | | | |
| HFC-1234ze(E), | 99.7 | 94.7 | 84.7 | 94.69 | 84.69 | 94.65 | 84.65 | 94.6 | 84.6 |
| wt % | | | | | | | | | |
| Ethanol, wt % | 0 | 5 | 15 | 5 | 15 | 5 | 15 | 5 | 5 |
| Oleic Acid, wt % | 0 | 0 | 0 | 0.01 | 0.01 | 0.05 | 0.05 | 0.1 | 0.1 |
| API, wt % | 0.3 | 0.3 | 0.3 | 0.3 | 0.3 | 0.3 | 0.3 | 0.3 | 0.3 |
| Test Results | | | | | | | | | |
| ADDU, % of | 110 | 80 | 73.4 | 73.8 | 81.8 | 88.6 | 87.9 | 96.2 | 73.9 |
| Target | | | | | | | | | |
| Standard | 45 | 45 | 17.7 | 10.2 | 8.9 | 8.5 | 6.9 | 5.4 | 18.7 |
| Deviation of | | | | | | | | | |
| ADDU % | | | | | | | | | |

As can be seen from the data above and in the charts in FIGS. 2A and 2B, the addition of 0.01 wt % of oleic acid to the formulation containing 5% by weight of ethanol unexpectedly produces a deterioration in the ADDU, decreasing it from 80 to 73.8. On the other hand, it has been unexpectedly found that increasing the oleic acid to 0.05% substantially increases the ADDU, to a % of target nearing 90% and up to greater than 95% for an oleic acid concentration of 0.1 wt. However, it is also seen (see FIG. 2B), that the standard deviation of the data increases as the amount of oleic acid increases to 0.1 wt %. For the 5% ethanol formulation, therefore, applicants have found that the ability to achieve an ADDU of 75% or higher while achieving a standard deviation below 15% requires an oleic acid concentration of greater than 0% but less than 0.1 wt %, and even more preferably from about 0.01 wt % to less than 0.1 wt % These results are unexpected.

With respect to the formulation which uses 15% ethanol, it is also seen from the data of this example that increasing the concentration of oleic acid above 0.05 wt % for the 15% ethanol formulation produces a deterioration in the ADDU, decreasing it from 87.9% to 73.9%. This result is unexpected.

### Comparative Example 2: Delivered Dose of Salbutamol Sulfate Suspensions in about 100% HFO-1234ze(E) with Oleic Acid

Comparative Example 1A is repeated except that the salbutamol sulfate (API) was suspended in HFC-1234ze(E) with the addition of 0.01, 0.05 and 0.1 wt % of oleic acid as indicated in Table C2 below. Each formulation was then loaded into an MDI with a target dosage delivery of 167 micrograms per actuation and then tested to determine the Average Delivered Dose Uniformity (ADDU) and Standard Deviation of ADDU (generally following the guidelines set forth in USP601—Inhalation and Nasal Drug Products: Aerosols, Sprays, and Powders—Performance Quality Tests The results of this test, together with results of Example C1A, are reported in Table ExC2.

TABLE ExC2

| Example No. | | | | |
|---|---|---|---|---|
| Carrier Components | ExC1A | ExC2A | ExC2B | ExC2C |
| HFC-1234ze(E), wt % | 99.7 | 99.69 | 99.65 | 99.6 |
| Oleic Acid, wt % | 0 | 0.01 | 0.05 | 0.1 |
| API, wt %* | 0.3 | 0.3 | 0.3 | 0.3 |
| ADDU, % of Target | 108.6 | 70.1 | 70.2 | 70.7 |
| Standard Deviation of | 45 | 16.8 | 6.9 | 25.5 |
| ADDU % | | | | |

As is seen from test result reported in Table ExC2 above, the use of a carrier comprising about 100% HFO-1234ze(E) and amounts of oleic acid of 0.05, 0.01 and 0.1 wt % in each case causes a substantial deterioration in the ADDU of the formulation to a value less than 75%.

### Comparative Example 3: Stability of Low Dose Salbutamol Sulfate Suspensions in about 100% HFO-1234ze(E) and HFC-134a

The formulation of Comparative Example 1A is tested for stability, together with a formulation made with a carrier consisting of HFC-134a, using the Turbiscan Stability Index (TSI), except that the concentration of the API is changed to 0.16 wt %. TSI is a test known to those skilled in the art as being determined as the cumulative sum of the change in light transmittance or backscattering throughout a sample of an API containing formulation at series of times after the formulation has been formed into a suspension, as would occur for example after an MDI containing the sample has been shaken. In this test, higher values indicate a larger change from the baseline (T=0) and more instability. More stable suspensions have lower TSI values. The TSI is determined according to the following calculation:

$$TSI ( t ) = \frac{1}{{N}_{h}} {\sum }_{{t}_{i} = 1}^{{t}_{max}} {\sum }_{{z}_{i} = {z}_{min}}^{{z}_{max}} \vert BST ( {t}_{i} , {z}_{i} ) - BST ( {t}_{i - 1} , {z}_{i} ) \vert$$

where:
tₘₐₓ=measurement time point
zₘᵢₙ=lower scanning height limit
zₘₐₓ=upper scanning height limit
Nₕ=(zₘₐₓ−zₘᵢₙ)/Δh=number of height positions
BST is the signal (backscattering (BS) if transmission (T) is <0.2%, or T otherwise).

The test is performed by adding the salbutamol sulfate in the indicated concentration into a clean, glass vial. When needed, the indicated amount of ethanol was then weighed into the same vial and capped with a crimped valve. HFO-1234ze(E) was then pressure filled through the valve to a final weight of 10 g. Vials were shaken vigorously for 10 seconds and then loaded into the Turbiscan Lab instrument (Formulaction, France). Samples were scanned at all heights every 25 seconds over a 5-minute period at 25° C. The TSI was calculated from the bottom of the vial to the meniscus of each formulation. Mean values were calculated from sample heights of 8 to 14 mm. Peak thickness was measured from the bottom of the sample to 3 mm, with an absolute ΔBS threshold of 6%. The results of this test are illustrated in FIG. ExC3.

As can be seen from the data obtained by this test, the use of a carrier consisting of HFO-1234ze produces a less stable solution at times of 270 seconds and 300 seconds compared to a carrier consisting of HFC-134a.

### Example 3: Stability of Low Dose Salbutamol Sulfate Suspensions in HFO-1234ze(E) and with 5% and 10% Ethanol

Comparative Example 3 is repeated using the same low dose of salbutamol (i.e., 0.16 wt %), except using as carrier blends consisting of: (1) about 95% by weight of HFO-1234ze(E) and 5% by weight of ethanol (Ex3A) and (2) about 90% by weight of HFO-1234ze(E) and 10% by weight of ethanol (Ex3B). The results of this test, together with the results from Comparative Example C3A, are provided in Table Ex3 below and are illustrated in FIG. Ex3.

TABLE Ex3
Salbutamol at 0.16 wt %

| Time, | 0 wt % EtOH | 5 wt % EtOH | 10 wt % EtOH |
|---|---|---|---|
| seconds | TSI | | |
| 0 | 0 | 0 | 0 |
| 30 | 3.4 | 7.8 | 6.6 |
| 60 | 6.8 | 8.9 | 7.2 |
| 90 | 9.7 | 9.8 | 7.7 |
| 120 | 11.9 | 10.4 | 8 |
| 150 | 13.6 | 10.8 | 8.2 |
| 180 | 15.1 | 11.1 | 8.4 |
| 210 | 16.3 | 11.2 | 8.5 |
| 240 | 17.4 | 11.4 | 8.5 |
| 270 | 18.6 | 11.5 | 8.6 |
| 300 | 19.9 | 11.7 | 8.6 |

As is seen from test result reported in Table Ex3 and in the chart of Figure Ex3, the addition of 5% and 10% ethanol improves the stability of the formulation at times greater than about 75 seconds compared to a carrier consisting of HFO-1234ze(E), but these formulations have a deteriorated stability compared to 100% HFO-1234ze(E) at times below 75 seconds. Importantly and unexpectedly, applicants have additionally found that for all times tested the formulation consisting of 10% ethanol and 90% HFO-1234ze(E) has a superior stability to the 5% ethanol formulation, with the superiority being the most significant at the time of 30 seconds. This is an important finding since the 30 second stability result is frequently considered as highly relevant since it corresponds generally to the period of time within which a user will use and MDI after shaking.

### Example 4: Stability of High Dose Salbutamol Sulfate Suspensions in HFO-1234ze(E) and with 5% and 10% Ethanol

Example 3 is repeated except using is a high dose of salbutamol (i.e., 0.3 wt %) and except with additional concentrations of ethanol in a carrier consisting of HFO-1234ze(E) and ethanol, as indicated in the Table Ex4 below and as illustrated in the chart of FIG. Ex4A.

TABLE Ex4
Salbutamol at 0.3 wt %

| | 0 wt % | 2.5 wt % | 3.5% | 5 wt % | 10 wt % | 15 wt % |
|---|---|---|---|---|---|---|
| Time, | EtOH | EtOH | EtOH | EtOH | EtOH | EtOH |
| seconds | TSI | | | | | |
| 0 | 0 | 0 | 0 | 0 | 0 | 0 |
| 30 | 16.2 | 17.9 | 13.4 | 13.4 | 11.6 | 7 |
| 60 | 19.1 | 21.4 | 14.7 | 14.7 | 13.1 | 9.4 |
| 90 | 21.2 | 24.1 | 15.7 | 15.7 | 14.2 | 11.1 |
| 120 | 22.8 | 26 | 16.5 | 16.5 | 15 | 12.4 |
| 150 | 23.9 | 27.4 | 17.1 | 17.2 | 15.6 | 13.2 |
| 180 | 24.6 | 28.3 | 17.4 | 17.4 | 16 | 13.8 |
| 210 | 25 | 28.8 | 17.6 | 17.6 | 16.2 | 14.2 |
| 240 | 25.3 | 29.2 | 17.7 | 17.7 | 16.3 | 14.5 |
| 270 | 25.6 | 29.6 | 17.8 | 17.8 | 16.5 | 14.9 |
| 300 | 26 | 30.1 | 17.9 | 18.2 | 16.6 | 15.4 |

As is seen from test result reported in Table Ex4 and the chart in Figure Ex. 4, the use of a carrier comprising 2% ethanol actually results in dramatic decrease in stability over the entire time range tested compared to 1234ze(E) alone, but unexpectedly increasing ethanol concentration to 3.5% and to 5% results in about the same dramatic increase in stability over the entire time range tested compared to 1234ze(E) alone and to the carrier comprising 2.5% of ethanol. This unexpected advantage continues for 10% ethanol and for 15% ethanol.

Furthermore, applicants have come to appreciate that the mean value of TSI measured mid sample, and the peak width measured at the bottom of the sample can give insight into particle size change and migration rate respectively. In particular, by measuring the mean ΔBS value through the middle of each sample gives insight on the size variation behavior (e.g., flocculation) of the formulations. Salbutamol particles suspended in HFO-1234ze(E) showed more rapid and greater size variation than in HFA-134a. The addition of increasing amounts of ethanol reduces the flocculation in HFO-1234ze(E), and without being bound to any theory of operation, it is believed that his effect results from breaking up the hydrophilic interactions of salbutamol, reducing the bonding forces between suspended particles. This data is illustrated in the chart in FIG. Ex4B.

In addition, migration rates were calculated by measuring the peak thickness of the change in backscattering at the bottom of each sample over the testing period and computing the slope for the linear portion, and these results are shown in the chart in FIG. 4C.

HFO-1234ze suspensions (bars on the right in FIG. 4C) had lower migration rates at 0% and 5% ethanol concentrations when compared to the corresponding HFA-134a formulation. However, at 10% ethanol, the migration rate in HFO-1234ze exceeds that of HFA-134a. This data suggests that there is a limit to the efficacy of ethanol in reducing migration rate and the trade-off with performance must be considered.

### Example 5: Stability of High Dose Salbutamol Sulfate Suspensions in HFO-1234ze(E) and with 15% Ethanol and Oleic Acid

Example 4 is repeated using 15% ethanol and a high dose of salbutamol (i.e., 0.3 wt %), except with addition of concentrations of oleic acid, as indicated in Table Ex5 below and as illustrated in the chart in FIG. Ex5.

TABLE Ex5
Salbutamol at 0.3 wt % and 15% Ethanol

| | | | | |
|---|---|---|---|---|
| | 0 wt % | 0.01 wt % | 0.05% | 0.1 wt % |
| Time, | Oleic Acid | Oleic Acid | Oleic Acid | Oleic Acid |

| | |
|---|---|
| seconds | TSI |

| | | | | |
|---|---|---|---|---|
| 0 | 0 | 0 | 0 | 0 |
| 30 | 7 | 9.7 | 5.3 | 4.9 |
| 60 | 9.4 | 11.7 | 9.2 | 8.5 |
| 90 | 11.1 | 13.2 | 10.7 | 10.1 |
| 120 | 12.4 | 14.2 | 11.7 | 11.3 |
| 150 | 13.2 | 14.9 | 12.4 | 12 |
| 180 | 13.8 | 15.5 | 12.9 | 12.5 |
| 210 | 14.2 | 15.8 | 13.2 | 12.8 |
| 240 | 14.5 | 16 | 13.4 | 13.1 |
| 270 | 14.9 | 16.3 | 13.6 | 13.3 |
| 300 | 15.4 | 16.5 | 13.8 | 13.8 |

As is seen from test result reported in Table Ex5 and the chart above, the use of a carrier comprising HFO-1234ze(E) and 15% ethanol and no oleic acid is unexpectedly more stable over the entire time range tested than with the use of 0.01 wt % oleic acid. However, increasing oleic acid concentration to 0.05 wt % and greater results unexpectedly in improved stability over the entire time range tested.

What is claimed is:

1. A pharmaceutical composition comprising:
a. an active pharmaceutical ingredient (API) comprising salbutamol;
b. from about 85% by weight to about 99% by weight of HFO-1234ze(E);
c. from 1% by weight to about 15% by weight of ethanol; and
d. from greater than 0.001% by weight to less than 0.1% by weight of oleic acid.

2. The pharmaceutical composition of claim 1 comprising from greater than 0.01% by weight to 0.05% by weight of oleic acid.

3. The pharmaceutical composition of claim 1 comprising:
a. from about 85% by weight to about 96.5% by weight of HFO-1234ze(E); and
b. from 3.5% by weight to about 15% by weight of ethanol.

4. The pharmaceutical composition of claim 1 comprising from greater than 0.01% by weight to 0.05% by weight of oleic acid.

5. The pharmaceutical composition of claim 1 comprising;
a. from about 85% by weight to about 90% by weight of HFO-1234ze(E); and
b. from 10% by weight to about 15% by weight of ethanol.

6. The pharmaceutical composition of claim 1 comprising;
a. from about 90% by weight to about 99% by weight of HFO-1234ze(E); and
b. from 1% by weight to about 10% by weight of ethanol.

7. The pharmaceutical composition of claim 1 comprising;
a. from about 95% by weight to about 99% by weight of HFO-1234ze(E); and
b. from 1% by weight to about 5% by weight of ethanol.

8. The pharmaceutical composition of claim 1 wherein said active pharmaceutical ingredient (API) consists essentially of salbutamol.

9. A pharmaceutical composition comprising:
a. an active pharmaceutical ingredient (API) comprising salbutamol;
b. from about 85% by weight to less than 99% by weight of HFO-1234ze(E); and
c. from greater than 5% by weight to about 15% by weight of ethanol.

10. The pharmaceutical composition of claim 9 comprising;
a. about 85% by weight of HFO-1234ze(E); and
b. about 15% by weight of ethanol.

11. The pharmaceutical composition of claim 9 comprising;
from greater than 0.01% by weight to less than 0.1% by weight of oleic acid.

12. The pharmaceutical composition of claim 9 comprising from greater than 0.001% by weight to less than about 0.05% by weight of oleic acid.

13. A method of forming pharmaceutical compositions having improved stability, comprising forming a carrier comprising not more than 99% by weight HFO-1234ze and from greater than about 1% by weight to less than about 10% by weight of ethanol, and suspending in said carrier an API comprising salbutamol.

14. The method of claim 13 wherein said suspension has improved migration rates compared to carrier comprising greater than about 10% of ethanol.

15. The method of claim 13 wherein said step of forming a carrier comprises forming a carrier comprising not more than 99% by weight HFO-1234ze and from greater than about 2.5% by weight to less than about 10% by weight of ethanol and wherein said suspending step comprises suspending salbutamol in said carrier in an amount of from 0.25 wt % to about 0.5 wt % based on the weight of the carrier and the salbutamol.

16. The method of claim 15 wherein said suspension has improved TSI compared to carrier comprising not greater than 2.5% by weight of ethanol.

17. The method of claim 13 wherein said step of forming a carrier comprises forming a carrier comprising not more than 99% by weight HFO-1234ze and from greater than about 2.5% by weight to less than about 10% by weight of ethanol.

18. The method of claim 13 wherein said suspending step comprises suspending salbutamol in said carrier in an amount of from 0.25 wt % to about 0.5 wt % based on the weight of the carrier and the salbutamol.

19. The method of claim 13 wherein said step of forming a carrier comprises forming a carrier comprising not more than 99% by weight HFO-1234ze and from greater than about 3.5% by weight to less than about 10% by weight of ethanol.

20. The method of claim 19 wherein said step of forming a carrier comprises forming a carrier comprising from greater than 0.01% by weight to 0.05% by weight of oleic acid.
